(19) United States
(12) Patent Application Publication
(10) Pub. No.: US 2011/0237925 A1
(43) Pub. Date: Sep. 29, 2011

(54) MICRONEEDLE ARRAY CHIP, DEVICE AND PATCH FOR TRANSDERMAL DRUG DELIVERY UTILIZING THE SAME, AND PREPARATION METHOD THEROF

(76) Inventors: Ruifeng Yue, Beijing (CN); Yan Wang, Beijing (CN)

(21) Appl. No.: 13/069,959

(22) Filed: Mar. 23, 2011

(30) Foreign Application Priority Data
Mar. 26, 2010 (CN) 201010180903.4

(51) Int. Cl. A61M 5/00; A61B 5/04; C23F 1/00; B32B 37/02; B32B 37/12; B32B 37/14; B23P 11/00; C25F 3/16

(52) U.S. Cl. 600/392; 604/173; 216/11; 156/278; 156/245; 156/256; 29/428; 205/640

(57) ABSTRACT

The invention discloses a microneedle array chip comprising metal microneedles and a substrate, wherein the microneedle consists of a needle head with a tip at its top, a needle bar and a needle seat, and is fixed onto the substrate via the needle seat; and the needle bar of the metal microneedle, having a cylindrical or conical shape, is inclined toward the substrate at a preset angle, and the needle head has a conical shape, or the upper surface of the tip is an oval plane or oval ring plane parallel to the substrate or inclined toward it at a preset acute angle. The metal microneedles in the microneedle array chip of the invention have firm structures to avoid fracture, and have sharp tips to facilitate puncturing. The maximal puncturing depth of the microneedles is easy to adjust and control. The microneedles in the array have good uniformity, and are safe and reliable to use. The hollow microneedles, like conventional syringe needles, have lateral openings, and thus can effectively avoid the blockage of the infusion poles by skin, thereby facilitating rapid diffusion and absorption of drugs, and resulting in significant therapeutic effects.

### RELATED APPLICATIONS

Priority is claimed to Chinese Patent Application No. 201010180903.4, filed Mar. 26, 2010, the disclosure of which is incorporated by reference in its entirety.

### FIELD OF THE INVENTION

The field of the present invention relates to medical and cosmetic devices, bio-medicine, and microfabrication technology, and in particular, to a microneedle array chip, a device for transdermal drug delivery, a patch for transdermal drug delivery, an electrode detection device based on microneedle array, and the preparation methods thereof.

### BACKGROUND

The therapeutic effect of a drug depends not only on the drug itself, but also on the drug delivery system, which plays an important role on the efficacy of the drug. Oral administration of biomacromolecular drugs, such as polypeptides, proteins, DNAs and vaccines, e.g. insulin, cannot achieve the desired therapeutic effect due to low bioavailability, which is caused by the enzymolysis in gastrointestinal tract, the first pass effect in liver, or the like. Subcutaneous injection, which can be used to conveniently and rapidly introduce a large dose of drug into human body and overcome the drawbacks of oral administration, has the limitations that it brings about pain to human body, injury at the injection site, and fear of needle to patients, especially child patients, and that it requires special technology, while cannot achieve sustained administration, and thus is not suitable for long-term administration. A transdermal drug delivery system, also known as a transdermal therapeutic system, refers to a preparation that allows a drug to penetrate through skin at a certain speed, enter systemic circulation via capillary vessels, and take effect. Compared to conventional administration methods, transdermal drug delivery has the advantages that it can produce a sustained, constant and controllable blood drug concentration, thus significantly increasing the activity of drugs having a short half life due to rapid metabolism in vivo, avoiding the interference of the first pass effect in liver and the factors of gastrointestinal tract, and minimizing toxic and side effects; that it is painless, noninvasive or minimally invasive; and that it can be conveniently performed by a patient himself/herself and be ceased at any time. Although transdermal drug delivery is attractive, there are only about 20 kinds of drugs entering the market of transdermal drug delivery in the world up to date, which mainly because of the blockage of the outmost layer of human skin, i.e., stratum corneum having a thickness of about 30 to 50 μm, resulting in that most drugs have a rate of transdermal permeation too low to satisfy the requirement of therapy. Presently, there are a lot of limitations on drugs that can be transdermally administered, for example, drugs for transdermal drug delivery should have a molecular weight lower than 500 D, a high liposolubility, a melting point lower than 150□, a therapeutic dosage lower than 20 mg/day, etc., and the passive transdermal drug delivery of polymeric and polar drugs is difficult to achieve. Nowadays, there are thousands of biomacromolecular drugs under development in the world, and the lack of suitable drug delivery system has become the major obstacle for the above drugs to smoothly enter clinic and the market to exhibit their optimal therapeutic effects.

In order to increase the permeability of skin, a plurality of methods, such as chemical penetration enhancer, iontophoresis, electroporation, etc., are employed. These methods have various degrees of limitations with respect to the drugs to be delivered, and some of them may cause serious toxic and side effects. In 1998, a solid silicon microneedle array prepared through micro-electromechanical system (MEMS) technology was first used in the field of transdermal drug delivery by the team of professor Prausnitz in US, and was found to be able to increase the transdermal permeability of the biomacromolecular model drug, calcein, by four orders of magnitude, which, therefore, leads to the extensive research of transdermal drug delivery utilizing microneedles in the world (S. Henry, D. V. McAllister, M. G. Allen, and M. R. Prausnitz. “Microfabricated microneedles, a novel approach to transdermal drug delivery”, J. Pharmaceut. Sci., 87(8) 922-925, 1998).

In a sense, administration via a microneedle array is a type of administration in between subcutaneous injection and administration via a transdermal patch. There are three layers of tissues in human skin: stratum corneum, active epidermis and dermis. The outmost layer is the about 30 to 50 μm-thick stratum corneum composed of compact keratinocytes, which has very low permeability to most of drugs, and is the main obstacle to the transdermal delivery of these drugs. Under stratum corneum is the about 50 to 100 μm-thick epidermis, which contains viable cells and a very small quantity of nerve tissues, but contains no blood vessel. Under epidermis is dermis, which is the main constituent of skin, and contains a large quantity of viable cells, nerve tissues and blood vessels. Conventional subcutaneous injection requires a needle, generally having an outer diameter of 0.4 to 3.4 mm, to puncture skin and get into muscles. This would undoubtedly allow touch of blood vessels by the needle, and nerve tissues may be injured, resulting in much pain to patient in addition to bleeding. Short and sharp microneedle arrays, which can be manufactured in large batch at low costs by utilizing MEMS technology, can instantaneously form a large quantity of micron-sized pore canals in stratum corneum and epidermis of skin to significantly increase drug permeability, and are theoretically suitable for any drug, including biomacromolecular drugs, without the limitations on the molecular weight, polarity, melting point, etc. of the drug. Administration via microneedles would not bring about pain and bleeding, since the administration sites are on the body surface without any touch of nerve tissues or blood vessels. In addition, administration via a microneedle array can be conveniently performed without professionals, and can be ceased at any time, and thus has high acceptance among patients.

Just like transdermal drug delivery, when a cosmetic product is applied by conventional methods, most of the active components in the cosmetic product cannot enter active epidermis and dermis due to the blockage of stratum corneum, thus resulting in insignificant cosmetic effects. If the skin is punctured by a microneedle array coated with the cosmetic product, or if the skin is punctured by a microneedle array before the application of the cosmetic product, the capability of the active components to penetrate stratum corneum and enter epidermis and dermis cells would be significantly increased, thus significantly improving the cosmetic effects. In addition, with the assistance of microneedles, a tattooing procedure, in which dyes are used instead of a cosmetic product, would undoubtedly become painless, safe and fast.

Currently, bioelectrodes have been widely used in modern clinical and biomedical examinations, such as electrocardiography (ECG), electroencephalography (EEG), and electrical impedance tomography (EIT). Undoubtedly, measuring electrodes have a great impact on the measuring accuracy and reliability of the measuring electronic devices used in these examinations. A microneedle array, which punctures stratum corneum of skin, can be used as the electrode to measure biopotential, thereby effectively avoiding the interference of the high impedance characteristic of stratum corneum of skin. Moreover, comparing with a common potential electrode, a microneedle array electrode needs neither skin preparation nor gel for electrolysis, and thus is suitable for long-term measurement. Accordingly, a measurement using a microneedle array electrode has better convenience and reliability, lower impedance, is supposed to have less electrochemical noise, and can record low biopotential with high-quality.

Microneedles can be classified into solid microneedles and hollow microneedles. Where solid microneedles are used, either of the following two administration methods is generally employed: 1) puncturing skin at the site to be treated with a microneedle array to form pore canals, and then applying a drug-containing patch onto the site; and 2) coating the surface of a microneedle array with a drug, and then puncturing the skin with the microneedle array to perform sustained drug release. Where hollow microneedles are used, drug delivery is generally performed by microinjection, which is suitable for liquid drugs and drugs with a high therapeutic dosage. In addition, hollow microneedles can be used in transdermal suction and detection of a trace amount of body fluid.

The materials for manufacturing microneedles include polymers, monocrystalline silicon, metals, etc. A serious problem of the available polymer microneedles is the insufficient strength of the material for readily puncturing stratum corneum of skin. Monocrystalline silicon is hard, and is prone to brittle fracture. Although monocrystalline silicon has been reported to have good biocompatibility in some literatures (e.g. Geoffrey Kotzar, Mark Freas, Phillip Abel, et al., Biomaterials, 23 (2002), 2737-2750), it is yet not a conventional medical material, and further study is necessary to determine whether it can be used in biomedical field. Although metals, which have been used for manufacturing needles for acupuncture or injection for thousands of years, is undoubtedly very safe, it is difficult to be processed into solid or hollow metal microneedle array chips in large batch at low costs by conventional precision machining processes.

### SUMMARY OF THE INVENTION

The object of the invention is to provide a metal microneedle array chip, a device for transdermal drug delivery, a patch for transdermal drug delivery, an electrode detection device based on microneedle array, and the preparation methods thereof.

The metal microneedles have firm structures, and have sharp tips to facilitate puncturing. The maximal puncturing depth of the microneedles is easy to adjust and control. The microneedles in the array have good uniformity, and are safe and reliable to use. The hollow microneedles, like conventional syringe needles, have lateral openings, and thus can effectively avoid the blockage of the infusion poles by skin, thereby facilitating rapid diffusion and absorption of drugs, and resulting in significant therapeutic effects.

In particular, the present invention provides:

(I). A microneedle array chip comprising metal microneedles and a substrate, wherein

the metal microneedle consists of a needle head with a tip at its top, a needle bar and a needle seat, and is fixed onto the substrate via the needle seat; and

the needle bar of the metal microneedle, having a cylindrical or conical shape, is inclined toward the substrate at a preset angle; and the needle head has a conical shape, or the upper surface of the tip is an oval plane or oval ring plane parallel to the substrate or inclined toward it at a preset acute angle.

(II). A method for preparing a microneedle array chip, comprising at least one of the following steps:

s101, inserting metal wire bars or metal capillaries into a substrate vertically or inclinedly at a preset angle, optionally allowing the former to penetrate the latter;

s102, cutting the metal wire bars or metal capillaries, and polishing the surfaces of the cuts at a direction parallel to or inclined toward the substrate to form oval tips; and

s103, dipping the microneedle array chip into a chemical or electrochemical polishing solution to subject the surfaces of the microneedles to chemical or electrochemical polishing.

(III). A patch for transdermal drug delivery, comprising the microneedle array chip according to the invention, and one or more films covering the microneedles or both the microneedles and the substrate of the microneedle array chip, wherein the films contain one or more materials comprising at least one drug or cosmetic component.

(IV). A method for preparing a patch for transdermal drug delivery, comprising the following steps:

s201, covering the microneedles or both the microneedles and the substrate of a microneedle array chip with a drug-containing film by means of dipping, fumigation, coating, or physical or chemical deposition; and

s202, bonding the back of the substrate to the central part of the adhesive-containing side of an adhesive tape, wherein protruding structures optionally exist on the edge of the substrate or in the peripheral part of the adhesive tape, and the surface of the remaining part of the said side of the adhesive tape and the surface of the chip are covered by a film that is easy to peel off.

(V) A device for transdermal drug delivery by means of a microneedle array chip, comprising a base having a manual handle in a planar or curved shape at its back, and a microneedle array chip permanently or detachably fixed to the front of the base, wherein

the size of the front of the base is less than, equal to or greater than the size of the microneedle array chip, and,

when the size of the front of the base is greater than the size of the microneedle array chip, the microneedle array chip is embedded in the center of the front of the base, and the edge of the front of the base and the substrate of the microneedle array are in a same surface, or the former is higher than the latter but lower than the needle head.

(VI). A method for preparing a device for transdermal drug delivery, comprising the following steps:

s301, forming a base by way of die casting, cutting, machining or bonding, wherein the size of the front of the base is less than, equal to or greater than the size of a microneedle array chip; and

s302, permanently or detachably fixing the microneedle array chip in the center of the front of the base by way of bonding, fusion, linking or mechanical compression.

(VII). An electrode detection device based on microneedle array, comprising:

a microneedle array chip according to the invention;

one or more unpatterned or patterned films of conductor materials, which connect with the metal microneedles of the microneedle array chip and external wires, and cover the back and/or front of the substrate of the microneedle array chip; and

an adhesive tape, wherein the back of the substrate of the microneedle array chip is bonded to the central part of the adhesive-containing side of the adhesive tape, and protruding structures optionally exist on the edge of the substrate or in the peripheral part of the adhesive tape, and the surface of the remaining part of the said side of the adhesive tape and the surface of the chip are covered by a film that is easy to peel off.

### BRIEF DESCRIPTION OF THE DRAWINGS

FIG. 1 is a schematic diagram showing the structure of a solid microneedle array chip according to an example of the invention, in which both the cylindrical needle bars and the needle seats are vertical to the substrate;

FIG. 2 is a schematic diagram showing the structure of a solid microneedle array chip according to an example of the invention, in which both the conical needle bars and the needle seats are vertical to the substrate;

FIG. 3 is a schematic diagram showing the structure of a solid microneedle array chip according to an example of the invention, in which both the conical needle bars and the needle seats are vertical to the substrate;

FIG. 4 is a schematic diagram showing the structure of a solid microneedle array chip according to an example of the invention, in which the upper surfaces of the tips of the needle heads are oval planes parallel to the substrate;

FIG. 5 is a schematic diagram showing the structure of a solid microneedle array chip according to an example of the invention, in which the upper surfaces of the tips of the needle heads are oval planes inclined toward the substrate and having more edges and corners thereon;

FIG. 6 is a schematic diagram showing the structure of a solid microneedle array chip according to an example of the invention, in which the needle heads have grooves thereon, and the upper surfaces of the tips of the needle heads are oval planes parallel to the substrate and having more edges and corners thereon;

FIG. 7 is a schematic diagram showing the structure of a hollow microneedle array chip according to an example of the invention, in which the upper surfaces of the tips of the needle heads are oval ring planes parallel to the substrate;

FIG. 8 is a schematic diagram showing the structure of a hollow microneedle array chip according to an example of the invention, in which the upper surfaces of the tips of the needle heads are oval ring planes parallel to the substrate and having more edges and corners thereon;

FIG. 9 is a schematic diagram showing the structure of a hollow microneedle array chip according to an example of the invention, in which the upper surfaces of the tips of the needle heads are oval ring planes inclined toward the substrate;

FIG. 10 is a sectional view showing the structure of a solid microneedle array chip according to an example of the invention, in which the needle bars are vertical to the substrate and the needle seats are inclined toward the substrate;

FIG. 11 is a sectional view showing the structure of a solid microneedle array chip according to an example of the invention, in which the needle bars are inclined toward the substrate and the needle seats are vertical to the substrate;

FIG. 12 is a sectional view showing the structure of a solid microneedle array chip according to an example of the invention, in which the needle bars and the needle seats are inclined toward the substrate;

FIG. 13 is a sectional view showing the structure of a solid microneedle array chip according to an example of the invention, in which the needle bars and the needle seats are inclined toward the substrate;

FIG. 14 is a sectional view showing the structure of a hollow microneedle array chip according to an example of the invention, in which the needle bars and the needle seats are vertical to the substrate;

FIG. 15 is a sectional view showing the structure of a hollow microneedle array chip according to an example of the invention, in which the needle bars are vertical to the substrate and the needle seats are inclined toward the substrate;

FIG. 16 is a sectional view showing the structure of a hollow microneedle array chip according to an example of the invention, in which the needle bars and the needle seats are vertical to the substrate;

FIG. 17 is a sectional view showing the structure of a hollow microneedle array chip according to an example of the invention, in which the needle bars and the needle seats are vertical to the substrate;

FIG. 18 is a sectional view showing the structure of a device for transdermal drug delivery based on a solid microneedle array chip according to an example of the invention;

FIG. 19 is a sectional view showing the structure of a device for transdermal drug delivery based on a solid microneedle array chip according to an example of the invention;

FIG. 20 is a sectional view showing the structure of a device for transdermal drug delivery based on a solid microneedle array chip according to an example of the invention;

FIG. 21 is a sectional view showing the structure of a device for transdermal drug delivery based on a solid microneedle array chip according to an example of the invention;

FIG. 22 is a sectional view showing the structure of a device for transdermal drug delivery based on a solid microneedle array chip according to an example of the invention;

FIG. 23A to FIG. 23D are the schematic diagrams showing the preparation process of a hollow microneedle array chip according to an example of the invention; and

FIG. 24A to FIG. 24E are the schematic diagrams showing the preparation process of a solid microneedle array chip according to an example of the invention.

In the drawings, “1” represents needle head; “2” represents needle bar; “3” represents needle seat; “4” represents substrate; “5” represents tip; “5a” represents edges and corners; “6” represents groove; “7” represents oval plane or oval ring plane; “8” represents via hole; “9” represents base; “10” represents protruding structure; and “11” represents manual handle.

### DETAILED DESCRIPTION OF THE INVENTION

In a first aspect, the present invention provides a microneedle array chip comprising metal microneedles and a substrate, wherein

the metal microneedle consists of a needle head with a tip at its top, a needle bar and a needle seat, and is fixed onto the substrate via the needle seat; and

the needle bar of the metal microneedle, having a cylindrical or conical shape, is inclined toward the substrate at a preset angle; and the needle head has a conical shape, or the upper surface of the tip is an oval plane or oval ring plane parallel to the substrate or inclined toward it at a preset acute angle.

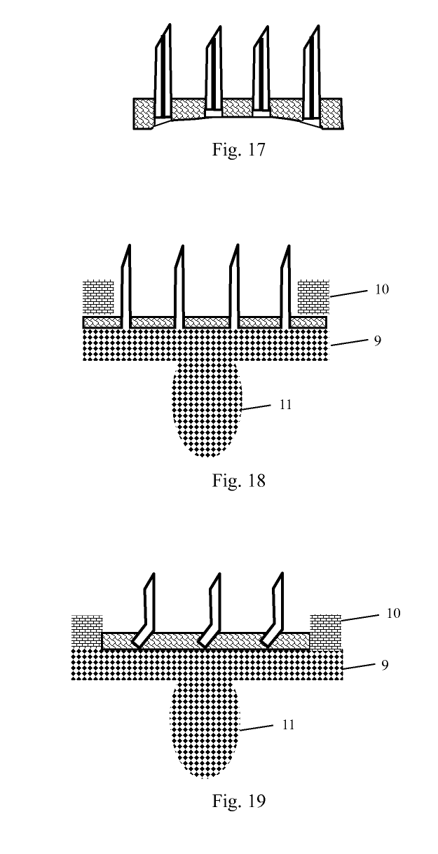

Preferably, the needle seat of the microneedle is embedded in or penetrates the substrate; the bottom of the needle seat and the bottom of the substrate are in a same surface, or the former protrudes from the latter, or the former is trapped in the latter. The substrate can be a planar one having either constant or varying thickness, and can also be a curved one as shown in FIG. 17.

Preferably, several pits exist in the oval plane of the tip, or at least a part of arc surface of the oval plane or oval ring plane is cut off so that more edges and corners appear thereon.

Preferably, the microneedle array, which is an array of solid metal microneedles or of hollow metal microneedles, or of a mixture of them, comprises at least two microneedles arranged on the substrate at a preset interval. The tips of the microneedles, either oval planes/oval ring planes or cones, are parallel, or are arranged to have one or more preset angles.

Preferably, the microneedles are solid metal microneedles or hollow metal microneedles, and the needle bars and needle seats of the metal microneedles are each inclined toward the substrate at their respective preset angles; the solid microneedles have grooves or pits, which serve as reservoirs of drugs, on the needle heads or needle bars, and the hollow metal microneedles have via holes between the needle seats and needle heads.

Preferably, the tips of the metal microneedles in the microneedle array chip are parallel or arranged at an angle to one another.

Preferably, the needle seat is inclined toward the substrate at a preset angle of 15° to 165°, and the preset angle at which the needle bar is inclined is 15° to 160° and more preferably 20° to 160°.

Preferably, the preset angle at which the needle seat is inclined is 15° to 90°, and the preset angle at which the needle bar is inclined is 15° to 150°.

Preferably, the metal microneedle is made of metal or alloy including, but not limited to, one or more of gold, silver, platinum, titanium, chromium, copper, aluminum, iron, nickel, tungsten, stainless steel, titanium alloy, aluminum alloy, nickel alloy, and copper alloy, and the surface of the metal microneedle is optionally covered by one or more films of dielectric materials and/or semiconductor materials and/or conductor materials, wherein the films of conductor materials include, but are not limited to, one or more of a metal film, an alloy film, and a film of an organometallic or inorganic compound.

Preferably, the film of conductor materials is a film of a metal selected from the group consisting of gold, titanium and platinum.

Preferably, the substrate is made of an insulator, semi-insulator or conductor material selected from the group consisting of medical plastic, polymer, synthetic resin, glass, rubber, latex and nonmetallic composite, or is composed of several layers, which are combined via an adhesive or a fixing element, and each of which is made of a material listed above.

Preferably, the radius of curvature, the thickness and the width of the tips are all in the range of 0.1 nm to 1,200 μm, more preferably, 1 nm to 500 μm, and most preferably, 5 nm to 350 μm.

Preferably, the needle bars have an outer diameter of 5 μm to 1,500 μm, more preferably, 20 μm to 1,000 μm, and most preferably, 50 μm to 250 μm, and a height of 10 μm to 10,000 μm, more preferably, 50 μm to 5,000 μm, and most preferably, 120 μm to 1,600 μm.

Preferably, the via holes of the hollow microneedles have an inner diameter of 1 μm to 1,000 μm, more preferably, 5 μm to 800 μm, and most preferably, 25 μm to 125 μm.

Preferably, the substrate, which is planar or curved, has a thickness of 20 μm to 8,000 μm, more preferably, 50 μm to 4,000 μm, and most preferably, 400 μm to 2,000 μm.

Preferably, the substrate is covered by one or more patterned or unpatterned films of dielectric materials and/or semiconductor materials and/or conductor materials on one or both sides thereof, and the films of conductor materials include, but are not limited to, one or more of a metal film, an alloy film, and a film of an organometallic or inorganic compound.

In a second aspect, the present invention provides a method for preparing a microneedle array chip, comprising at least one of the following steps:

s101, inserting metal wire bars or metal capillaries into a substrate vertically or inclinedly at a preset angle, optionally allowing the former to penetrate the latter;

s102, cutting the metal wire bars or metal capillaries, and polishing the surfaces of the cuts at a direction parallel to or inclined toward the substrate to form oval tips; and

s103, dipping the microneedle array chip into a chemical or electrochemical polishing solution to subject the surfaces of the microneedles to chemical or electrochemical polishing.

Preferably, the method additionally comprises the following steps between steps s102 and s103:

s102a, coating the substrate on the side having the metal wire bars or metal capillaries with a masking film, and forming predesigned masking film patterns on one end of the metal wire bars or metal capillaries by means of pattern transfer technology; and

s102b, chemically or electrochemically etching the metal wire bars or metal capillaries unprotected by the masking film, and removing the masking film after solid metal microneedles having needle heads with pits or grooves, needle bars with grooves towards the needle heads or with pits, and tips with more edges and corners, or hollow metal microneedles having tips with more edges and corners are formed.

Preferably, the method additionally comprises the following steps after step s103:

s103a, covering the substrate with a film of dielectric materials and/or a film of semiconductor materials and/or a film of conductor materials;

s103b, forming a masking film on the substrate, and forming predesigned masking film patterns on the substrate by means of pattern transfer technology or screen printing technology; and

s103c, chemically or electrochemically etching the film of dielectric materials and/or the film of semiconductor materials and/or the film of conductor materials unprotected by the masking film on the substrate, and removing the masking film after the predesigned patterns are formed.

In a third aspect, the present invention provides a patch for transdermal drug delivery, comprising the above-mentioned microneedle array chip, and one or more films covering the microneedles or both the microneedles and the substrate of the microneedle array chip, wherein the films contain one or more materials comprising at least one therapeutic, diagnostic, or prophylactic drug, or a cosmetic component for skin care, whitening and/or skin nourishment.

Preferably, the material contained in the films is in the form of a solid, a liquid, microparticles, a sol, a gel, a plaster, an ointment, or a mixture of two or more of them, which may further comprise adjuvants such as viscosity improver and solvent.

In a fourth aspect, the present invention provides a method for preparing a patch for transdermal drug delivery, comprising the following steps:

s201, covering the microneedles or both the microneedles and the substrate of a microneedle array chip with a drug-containing film by means of dipping, fumigation, coating, or physical or chemical deposition; and

s202, bonding the back of the substrate to the central part of the adhesive-containing side of an adhesive tape, wherein protruding structures optionally exist on the edge of the substrate or in the peripheral part of the adhesive tape, and the surface of the remaining part of the said side of the adhesive tape and the surface of the chip are covered by a film that is easy to peel off.

In the method according to the fourth aspect of the invention, the microneedles are preferably solid ones that have been coated with a surfactant for improving their soakage and adhesion of the drug before step s201, and the protruding structures can be made of plastics, rubbers or cloth.

In a fifth aspect, the present invention provides a device for transdermal drug delivery by means of a microneedle array chip, comprising a base having a manual handle in a planar or curved shape at its back, and a microneedle array chip permanently or detachably fixed to the front of the base, wherein the size of the front of the base is less than, equal to or greater than the size of the microneedle array chip, and when the size of the front of the base is greater than the size of the microneedle array chip, the microneedle array chip is embedded in the center of the front of the base, and the edge of the front of the base and the substrate of the microneedle array are in a same surface, or the former is higher than the latter but lower than the needle head.

Optionally, the device have permanent or detachable protruding structures on or around the edge of the substrate, which are provided for limiting the largest depth of the microneedles into skin when applying them.

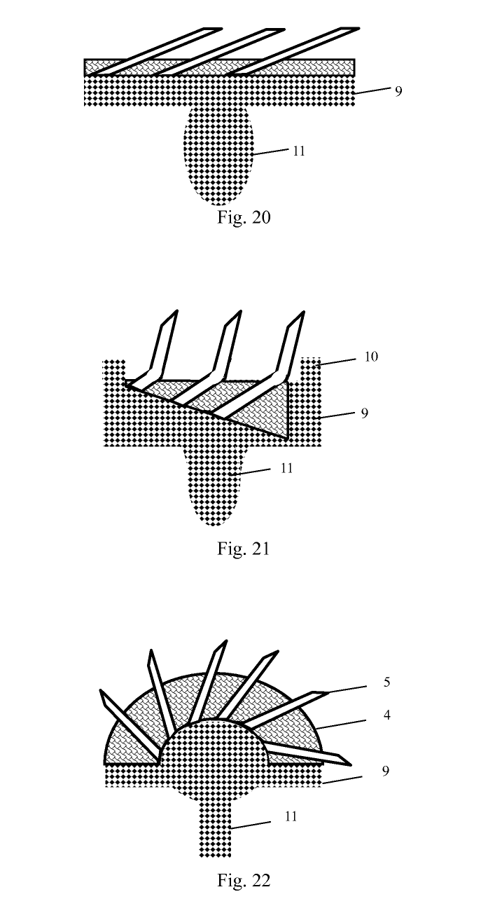

Preferably, when the microneedles of the microneedle array chip are solid microneedles, the device have, on the back of the base, a planar or curved handle 11 that is convenient to operate manually. FIG. 18 to FIG. 22 are sectional views showing the structures of several devices for transdermal drug delivery based on solid microneedle array chips according to the examples of the invention, in which the substrate of the array chip is planar or curved while the shape of the base at the side contacting the substrate is adapted to that of the substrate. Prominent structures can be arranged on or around the edge of the substrate of the array chip, where necessary.

Preferably, when the microneedles of the microneedle array chip are hollow microneedles, the microneedle array chip and the base are combined such that a structure having an inner cavity is formed, and the base has thereon a channel and an inlet for adding a drug solution into the inner cavity or an element for piercing the package of the drug solution inside the inner cavity.

Preferably, the base has a propelling unit for pressing the inner cavity, which unit is connected to the back of the base via a pipe or is integrated with the latter. The propelling unit works by pushing a piston or changing the shape of an elastic diaphragm by means of manual, electric or thermal operation, or the mechanical motion of shape memory alloy, spring or reed, or other methods. Also, the propelling unit may be a minipump that works manually, piezoelectrically, electrically, electromagnetically or the like. An intelligent system for controlling the propelling speed, time and monitoring the residual amount of drug solution can be integrated in the base or in the propelling unit outside the base, where necessary.

The base is preferably made of one or more materials selected from the group consisting of plastic, polymer, synthetic resin, ceramic, glass, rubber, latex, metal and composite, and adhesives or fixing elements are optionally used to combine the materials.

In a sixth aspect, the present invention provides a method for preparing a device for transdermal drug delivery, comprising the following steps:

s301, forming a base by way of die casting, cutting, machining or bonding, wherein the size of the front of the base is less than, equal to or greater than the size of a microneedle array chip; and

s302, permanently or detachably fixing the microneedle array chip to the center of the front of the base by way of bonding, fusion, linking or mechanical compression.

Preferably, when the microneedles of the microneedle array chip are hollow microneedles, the base formed in step s301 has thereon a channel and an inlet for adding a drug solution into the inner cavity, or an element for piercing the package of the drug solution inside the inner cavity.

Preferably, the method additionally comprises the following step between steps s301 and s302:

s301a, connecting a propelling unit for pressing the inner cavity to the back of the base via a pipe, or integrating the former with the latter.

Preferably, the propelling unit for pressing the inner cavity is mounted in the base by way of bonding, linking, welding, screw or bayonet, or bolt fixation, or is connected to the base via a pipe.

In a seventh aspect, the present invention provides an electrode detection device based on microneedle array, comprising:

a microneedle array chip according to the first aspect of the invention;

one or more unpatterned or patterned films of conductor materials, which connect with the metal microneedles of the microneedle array chip and external wires, and cover the back and/or front of the substrate of the microneedle array chip; and

an adhesive tape, wherein the back of the substrate of the microneedle array chip is bonded to the central part of the adhesive-containing side of the adhesive tape, and protruding structures optionally exist on the edge of the substrate or in the peripheral part of the adhesive tape, and the surface of the remaining part of the said side of the adhesive tape and the surface of the chip are covered by a film that is easy to peel off.

The above technical solutions have the following advantages: 1) the metal microneedles have firm structures to avoid fracture, and have sharp tips to facilitate puncturing; 2) the hollow microneedles, like conventional syringe needles, have lateral openings, and thus can effectively avoid the blockage of the infusion poles by skin, thereby facilitating rapid diffusion and absorption of drugs; 3) the microneedle array chip and the relevant devices can be manufactured in batches from conventional medical materials using well-developed processes at low costs, high yields and with good reproducibility; 4) the maximal puncturing depth of the microneedles is easy to adjust and control, and the microneedles in the array have good uniformity, are safe, reliable and durable to use; and 5) the microneedle array chip and the relevant devices are very applicable for the transdermal delivery of biomacromolecular drugs (e.g., polypeptides, proteins, DNAs and vaccines, etc.) and cosmetic products, and for the development of novel transdermal preparations of existed drugs, and have a very wide application prospect.

### EXAMPLES

Next, the specific embodiments of the invention will be described in detail by reference to the drawings and the examples. The examples are provided for the purpose of illustration only, and should not be construed as limiting the scope of the invention, which is defined by the appended claims.

### Example 1

### Preparation of a Solid Stainless Steel Microneedle Array Chip

Solid stainless steel needles with conical tips and having an outer diameter of 300 μm at most were vertically inserted at a preset interval into a 2 mm-thick solution of methyl methacrylate prepolymers for forming a poly(methyl methacrylate) substrate, until the distance between the tip of the needle and the upper surface of the substrate reaches the preset value (0.5 mm to 1.6 mm) After heating to polymerize and cure, an embryonic form of a microneedle array chip was formed. The lower surface of the substrate was then polished, forming a solid stainless steel microneedle array chip as shown in FIG. 1, 2 or 3.

### Example 2

### Preparation of a Solid Stainless Steel Microneedle Array Chip

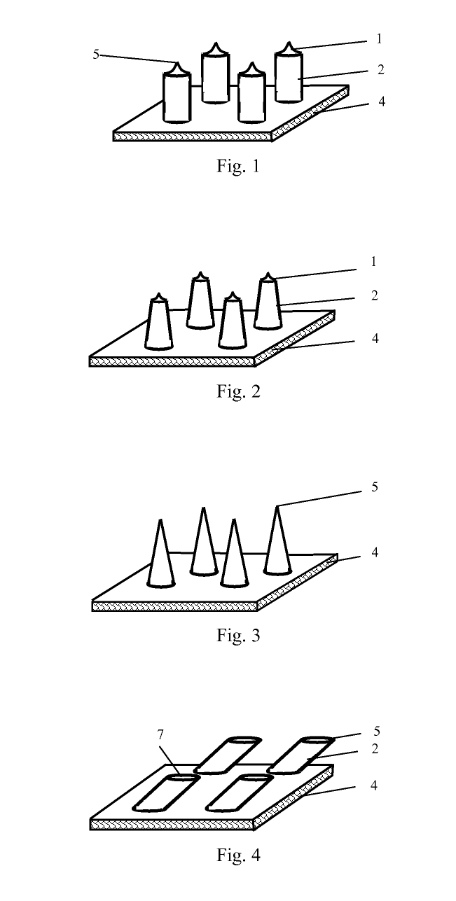

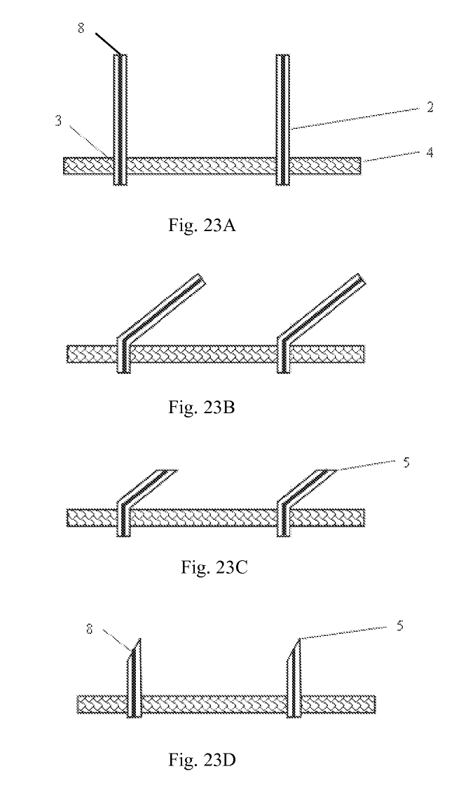

Solid stainless steel bars having an outer diameter of 200 μm were inserted vertically or inclinedly at a preset angle, at a preset interval, into a 2 mm-thick solution of methyl methacrylate prepolymers for forming a poly(methyl methacrylate) substrate. After heating to polymerize and cure, the bars were cut off at the points 0.5 mm to 3 mm away from the upper surface of the substrate, forming an embryonic form of a microneedle array chip (FIG. 23A). The stainless steel bars above the upper surface of the substrate were pushed, where necessary, to adjust the angles between them and the substrate (FIG. 23B). The cuts were then polished at a direction parallel to or inclined at a preset angle toward the upper surface of the substrate such that the desired height of needle was obtained and oval tips were formed (FIG. 23C). Finally, the stainless steel bars were further pushed, where necessary, to adjust the angles between them and the substrate (FIG. 23D), forming a solid stainless steel microneedle array chip as shown in FIG. 4, 10, 11, 12 or 13.

### Example 3

### Preparation of a Hollow Stainless Steel Microneedle Array Chip

With a method similar to that described in Example 2, except using hollow stainless steel tubes having an outer diameter of 200 μm instead of the solid stainless steel bars, a hollow stainless steel microneedle array chip as shown in FIG. 7, 9, 14 or 15 was formed.

### Example 4

### Preparation of a Solid Stainless Steel Microneedle Array Chip

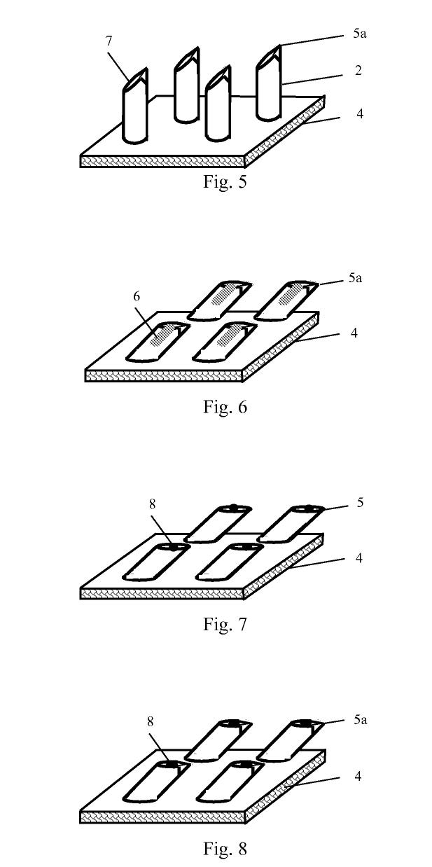

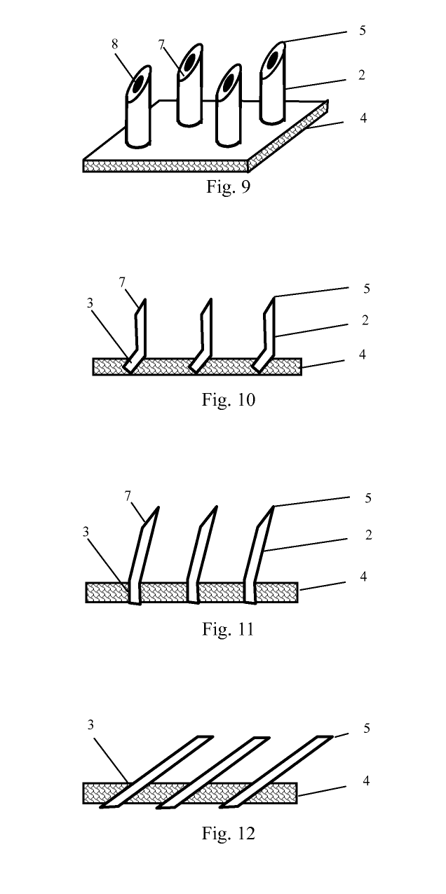

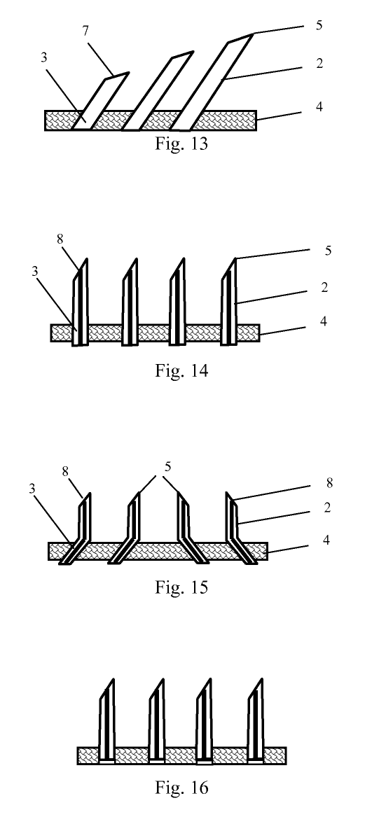

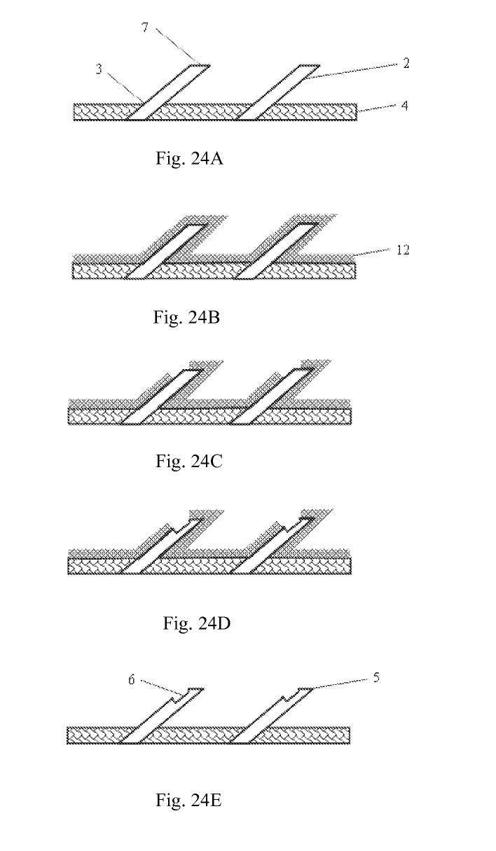

Solid stainless steel bars having an outer diameter of 200 μm were inserted vertically or inclinedly at a preset angle, at a preset interval, into a 2 mm-thick solution of epoxy resin prepolymers for forming an epoxy resin substrate. After heating to polymerize and cure, an embryonic form of a microneedle array chip was formed. The back of the substrate of the chip was polished so that all the bars were exposed from this side. The cuts of the bars at the side of the upper surface of the substrate were then polished at a direction parallel to or inclined at a preset angle toward the upper surface of the substrate such that the desired height of needle was obtained (FIG. 24A). The surfaces of the substrate and the bars at the side of the upper surface of the substrate were coated with photoresist layer 12 (FIG. 24B). Photoresist layer 12 was subjected to selective exposure and development by means of pattern transfer technology commonly used in microelectronic processes to form photoresist patterns on the bars (FIG. 24C). With photoresist layer 12 acting as a masking film, the bars at the side of the upper surface of the substrate were subjected to selective chemical etching (chemical etching solution: an aqueous ferric chloride solution having a Baume degree of 35-45; etching period: 20 min to 60 min) or electrochemical etching (procedures: dipping the microneedle array-containing side of the substrate of the chip in 1000 mL of an electrochemical etching solution containing 16-38 wt % ferric chloride, 1-10 vol % hydrochloric acid, 1-10 vol % nitric acid, and 0.1-0.5 wt % potassium dichromate; dipping the other side of the substrate of the chip in a conductive aqueous solution containing 0.1-0.5 wt % NaCl; introducing a graphite electrode in each of the two solutions, and applying a direct voltage of 5-25 V between the graphite electrodes, wherein positive potential was first applied to the electrode in the etching solution for 2 min to 10 min, and then negative potential for 20 min to 50 min), thus forming tips with grooves, pits or more edges and corners at the end of the bars (FIG. 24D). The photoresist was removed with acetone. Then the chip was subjected to chemical etch polishing (chemical etch polishing solution: an aqueous solution containing 80-120 g/L hydrochloric acid, 50-60 g/L nitric acid and 150-200 g/L phosphoric acid; etching period: 2 min to 10 min) or electrochemical etch polishing (procedures: dipping the microneedle array-containing side of the substrate of the chip in an electrochemical etch polishing solution containing 600 mL/L phosphoric acid and 300 mL/L sulfuric acid, in which a lead positive electrode was introduced; dipping the other side of the substrate of the chip in a conductive aqueous solution containing 0.1-0.5 wt % NaCl, in which a lead negative electrode was introduced; and applying a direct voltage of 8-10 V between the two electrodes for 2 min to 10 min), forming a solid microneedle array chip as shown in FIG. 5 or 6.

### Example 5

### Preparation of a Hollow Stainless Steel Microneedle Array Chip

With a method similar to that described in Example 4, except using hollow stainless steel tubes having an outer diameter of 200 μm instead of the solid stainless steel bars, a hollow stainless steel microneedle array chip as shown in FIG. 8 or 16 was formed.

### Example 6

### Preparation of a Device for Transdermal Drug Delivery Containing a Solid Microneedle Array Chip

Polyethylene base 9 and handle 11 as shown in FIG. 18 to FIG. 22 were manufactured using a plastic injection molding machine. The substrate of a solid microneedle array chip was cut into a desired size by means of a conventional cutter. The chip was then directly bonded to base 9. Finally, rubber layer 10 having an appropriate thickness, which was provided for limiting the maximal puncturing depth of the microneedles into skin, was bonded to the edge of the substrate or the edge of the base in the periphery of the chip, forming a device for transdermal drug delivery as shown in FIG. 18, 19, 20, 21 or 22.

While the invention has been described with respect to the specific embodiments, it should be recognized that various modifications and changes may be made by those skilled in the art, which also fall within the scope of the invention as defined in the appended claims.

What is claimed is:

1. A microneedle array chip comprising metal microneedles and a substrate, wherein
the metal microneedle consists of a needle head with a tip at its top, a needle bar and a needle seat, and is fixed onto the substrate via the needle seat; and
the needle bar of the metal microneedle, having a cylindrical or conical shape, is inclined toward the substrate at a preset angle; and the needle head has a conical shape, or the upper surface of the tip is an oval plane or oval ring plane parallel to the substrate or inclined toward it at a preset acute angle.

2. The microneedle array chip according to claim 1, wherein the needle seat of the metal microneedle is embedded in or penetrates the substrate; the bottom of the needle seat and the bottom of the substrate are in a same surface, or the former protrudes from the latter, or the former is trapped in the latter.

3. The microneedle array chip according to claim 1, wherein several pits exist in the oval plane of the tip, or at least a part of arc surface of the oval plane or oval ring plane is cut off so that more edges and corners appear thereon.

4. The microneedle array chip according to claim 1, wherein the microneedle array, which is an array of solid metal microneedles or of hollow metal microneedles, or of a mixture of them, comprises at least two microneedles arranged on the substrate at a preset interval.

5. The microneedle array chip according to claim 4, wherein the microneedles are solid metal microneedles or hollow metal microneedles, and the needle bars and needle seats of the metal microneedles are each inclined toward the substrate at their respective preset angles; the solid metal microneedles have grooves or pits on the needle heads or needle bars, and the hollow metal microneedles have via holes between the needle seats and needle heads.

6. The microneedle array chip according to claim 5, wherein the tips of the metal microneedles in the microneedle array chip are parallel or arranged at an angle to one another.

7. The microneedle array chip according to claim 5, wherein
the radius of curvature, the thickness and the width of the tips are all in the range of 0.1 nm to 1,200 μm;
the needle bars have an outer diameter of 5 μm to 1,500 μm and a height of 10 μm to 10,000 μm;
the via holes of the hollow microneedles have an inner diameter of 1 μm to 1,000 μm; and
the substrate, which is planar or curved, has a thickness of 20 μm to 8,000 μm.

8. The microneedle array chip according to claim 7, wherein
the radius of curvature of the tips is in the range of 5 nm to 350 μm;
the needle bars have an outer diameter of 20 μm to 1,000 μm and a height of 50 μm to 5,000 μm; and
the via holes of the hollow metal microneedles have an inner diameter of 5 μm to 800 μm.

9. The microneedle array chip according to claim 7, wherein the substrate is covered by one or more patterned or unpatterned films of dielectric materials and/or semiconductor materials and/or conductor materials on one or both sides thereof, and the films of conductor materials include, but are not limited to, one or more of a metal film, an alloy film, and a film of an organometallic or inorganic compound.

10. The microneedle array chip according to claim 1, wherein the needle seat is inclined toward the substrate at a preset angle of 15° to 165°, and the preset angle at which the needle bar is inclined is 15° to 160°.

11. The microneedle array chip according to claim 10, wherein the preset angle at which the needle seat is inclined is 15° to 90°, and the preset angle at which the needle bar is inclined is 15° to 150°.

12. The microneedle array chip according to claim 1, wherein the metal microneedle is made of metal or alloy including, but not limited to, one or more of gold, silver, platinum, titanium, chromium, copper, aluminum, iron, nickel, tungsten, stainless steel, titanium alloy, aluminum alloy, nickel alloy, and copper alloy, and the surface of the metal microneedle is optionally covered by one or more films of dielectric materials and/or semiconductor materials and/or conductor materials, wherein the films of conductor materials include, but are not limited to, one or more of a metal film, an alloy film, and a film of an organometallic or inorganic compound.

13. The microneedle array chip according to claim 12, wherein the film of conductor materials is a film of a metal selected from the group consisting of gold, titanium and platinum.

14. The microneedle array chip according to claim 1, wherein the substrate is made of an insulator, semi-insulator or conductor material selected from the group consisting of medical plastic, polymer, synthetic resin, glass, rubber, latex and nonmetallic composite, or is composed of several layers, which are combined via an adhesive or a fixing element, and each of which is made of a material listed above.

15. A patch for transdermal drug delivery, comprising the microneedle array chip according to claim 1, and one or more films covering the microneedles or both the microneedles and the substrate of the microneedle array chip, wherein the films contain one or more materials comprising at least one drug or cosmetic component.

16. The patch for transdermal drug delivery according to claim 15, wherein the material is in the form of a solid, a liquid, microparticles, a sol, a gel, a plaster, an ointment, or a mixture of two or more of them.

17. An electrode detection device based on microneedle array, comprising:
a microneedle array chip as defined in claim 1;
one or more unpatterned or patterned films of conductor materials, which connect with the metal microneedles of the microneedle array chip and external wires, and cover the back and/or front of the substrate of the microneedle array chip; and
an adhesive tape, wherein the back of the substrate of the microneedle array chip is bonded to the central part of the adhesive-containing side of the adhesive tape, and protruding structures optionally exist on the edge of the substrate or in the peripheral part of the adhesive tape, and the surface of the remaining part of the said side of the adhesive tape and the surface of the chip are covered by a film that is easy to peel off.

18. A method for preparing a microneedle array chip, comprising at least one of the following steps:
s101, inserting metal wire bars or metal capillaries into a substrate vertically or inclinedly at a preset angle, optionally allowing the former to penetrate the latter;
s102, cutting the metal wire bars or metal capillaries, and polishing the surfaces of the cuts at a direction parallel to or inclined toward the substrate to form oval tips; and
s103, dipping the microneedle array chip into a chemical or electrochemical polishing solution to subject the surfaces of the microneedles to chemical or electrochemical polishing.

19. The method according to claim 18, additionally comprising the following steps between steps s102 and s103:
s102a, coating the substrate on the side having the metal wire bars or metal capillaries with a masking film, and forming predesigned masking film patterns on one end of the metal wire bars or metal capillaries by means of pattern transfer technology; and
s102b, chemically or electrochemically etching the metal wire bars or metal capillaries unprotected by the masking film, and removing the masking film after solid metal microneedles having needle heads with pits or grooves, needle bars with grooves towards the needle heads or with pits, and tips with more edges and corners, or hollow metal microneedles having tips with more edges and corners are formed.

20. The method according to claim 19, additionally comprising the following steps after step s103
s103a, covering the substrate with a film of dielectric materials and/or a film of semiconductor materials and/or a film of conductor materials;
s103b, forming a masking film on the substrate, and forming predesigned masking film patterns on the substrate by means of pattern transfer technology or screen printing technology; and
s103c, chemically or electrochemically etching the film of dielectric materials and/or the film of semiconductor materials and/or the film of conductor materials unprotected by the masking film on the substrate, and removing the masking film after the predesigned patterns are formed.

21. A method for preparing a patch for transdermal drug delivery, comprising the following steps:
s201, covering the microneedles or both the microneedles and the substrate of a microneedle array chip with a drug-containing film by means of dipping, fumigation, coating, or physical or chemical deposition; and
s202, bonding the back of the substrate to the central part of the adhesive-containing side of an adhesive tape, wherein protruding structures optionally exist on the edge of the substrate or in the peripheral part of the adhesive tape, and the surface of the remaining part of the said side of the adhesive tape and the surface of the chip are covered by a film that is easy to peel off.

22. The device for transdermal drug delivery according to claim 21, wherein the microneedles of the microneedle array chip are hollow microneedles, the microneedle array chip and the base are combined such that a structure having an inner cavity is formed, and the base has thereon a channel and an inlet for adding a drug solution into the inner cavity or an element for piercing the package of the drug solution inside the inner cavity.

23. The device for transdermal drug delivery according to claim 22, wherein
the base has a propelling unit for pressing the inner cavity, which unit is connected to the back of the base via a pipe or is integrated with the latter, and
the base is made of one or more materials selected from the group consisting of plastic, polymer, synthetic resin, ceramic, glass, rubber, latex, metal and composite, and adhesives or fixing elements are optionally used to combine the materials.

24. A device for transdermal drug delivery by means of a microneedle array chip, comprising a base having a manual handle in a planar or curved shape at its back, and a microneedle array chip permanently or detachably fixed to the front of the base, wherein
the size of the front of the base is less than, equal to or greater than the size of the microneedle array chip, and,
when the size of the front of the base is greater than the size of the microneedle array chip, the microneedle array chip is embedded in the center of the front of the base, and the edge of the front of the base and the substrate of the microneedle array are in a same surface, or the former is higher than the latter but lower than the needle head.

25. A method for preparing a device for transdermal drug delivery, comprising the following steps:
s301, forming a base by way of die casting, cutting, machining or bonding, wherein the size of the front of the base is less than, equal to or greater than the size of a microneedle array chip; and
s302, permanently or detachably fixing the microneedle array chip in the center of the front of the base by way of bonding, fusion, linking or mechanical compression.

26. The method according to claim 25, wherein the microneedles of the microneedle array chip are hollow microneedles, and the base formed in step s301 has thereon a channel and an inlet for adding a drug solution into the inner cavity, or an element for piercing the package of the drug solution inside the inner cavity.

27. The method according to claim 25, additionally comprising the following step between steps s301 and s302:
s301a, connecting a propelling unit for pressing the inner cavity to the back of the base via a pipe, or integrating the former with the latter.

28. The method according to claim 27, wherein the propelling unit for pressing the inner cavity is mounted in the base by way of bonding, linking, welding, screw or bayonet, or bolt fixation, or is connected to the base via a pipe.
